(19) United States
(12) Patent Application Publication
(10) Pub. No.: US 2009/0041887 A1
(43) Pub. Date: Feb. 12, 2009

(54) High Protein Soybean Meal

(75) Inventors: Jihong Liang, Chesterfield, MO (US); Fang Chi, Libertyville, IL (US); Douglas C. Kotowski, St. Louis Park, MN (US)

Correspondence Address: SONNENSCHEIN NATH & ROSENTHAL LLP, P.O. BOX 061080, SOUTH WACKER DRIVE STATION, SEARS TOWER, CHICAGO, IL, 60606, US

(73) Assignees: RESNESSEN LLC, Deerfield, IL (US); MONSANTO TECHNOLOGY, LLC, St. Louis, MO (US); CARGILL, INCORPORATED, Wayzata, MN (US)

(21) Appl. No.: 10/587,114

(22) PCT Filed: Jan. 26, 2005
(86) PCT No.: PCT/US2005/002503

§ 371 Date: Aug. 29, 2008

Related U.S. Application Data
(60) Provisional application No. 60/539,168, filed on Jan. 26, 2004.

(51) Int. Cl. A23K 1/14; C07K 14/415

(52) U.S. Cl. 426/2; 426/656; 530/378

(57) ABSTRACT

A high protein soybean meal is disclosed. The soybean meal is generated from soybeans that are capable of commercial yields, wherein the meal comprises at least 58% protein on a dry weight basis. The soybean meal of the present invention may also be generated from soybeans comprising a mean whole seed total protein plus oil content of greater than about 64%, on a dry weight basis, wherein the soybean has a yield, under standard agronomic conditions, of at least 30 bushels per acre. Also disclosed is an animal feed containing the soybean meal of the present invention.

This application claims priority to U.S. Provisional Application No. 60/539,168 filed Jan. 26, 2004, the disclosure of which is incorporated herein by reference in its entirety.

The present invention relates to the area of animal nutrition and specialty feeds. In particular the present invention relates to a high protein soybean meal suitable for use as an ingredient in animal feeding operations.

Soybeans are a major agricultural commodity in many parts of the world, and they are the source of many useful products for both human and animal consumption. Two of the more important commercial products obtained from soybeans are soybean oil and soybean meal. Soybean oil is used as an energy source in animal feeds although its primary use is for human consumption. Soybean meal is used primarily as a component in animal feed.

Commercial soybean meals are a good source of amino acids in poultry diets as they are relatively high in protein when compared to other grain sources such as corn. A soybean meal having a higher protein content would be desirable (Edwards et al., Poultry Sci., 79:525-527 (2000)). There is a limitation, however, on total endogenous protein content in commercial soybean meal because commercial soybeans are typically about 41% protein on a dry matter basis. Substantially higher protein content in soybeans, such as in excess of 55% on a dry weight basis, has been uniformly associated with poor agronomic qualities, such as poor yield. See for example, Wehrmann et al., Crop Sci., 27:927-931 (1987) and Simpson and Wilcox, Crop Sci., 23:1077-1081 (1983). Additionally, use of exogenous protein sources to supplement soybean meals adds cost and formulation problems.

Therefore, it would be desirable to have a soybean meal having a higher endogenous protein content that is derived from soybeans that have favorable agronomic qualities.

### SUMMARY OF THE INVENTION

The present invention provides answers to the needs articulated above. In particular, the present invention provides a soybean meal, generated from a soybean capable of commercial yields, comprising at least about 58% protein on a dry weight basis. In another embodiment, the present invention provides a soybean meal, generated from a soybean capable of commercial yields, comprising at least about 60% protein on a dry weight basis. In a further embodiment, the present invention provides a soybean meal, generated from a soybean capable of commercial yields, comprising at least about 62% protein on a dry weight basis.

In yet another aspect, the soybean has an actual grain yield, under standard agronomic practices, of at least about 30 bushels per acre. In a further aspect, the soybean has a comparative yield of at least about 67% of an agronomically elite variety.

The present invention further provides a feed containing the soybean meal, generated from a soybean capable of commercial yields, comprising at least about 58% protein on a dry weight basis.

The present invention further provides a feed containing the soybean meal generated from a soybean, comprising at least about 58% protein on a dry weight basis, wherein the soybean has a grain yield, under standard agronomic practices, of at least about 30 bushels per acre.

In a further aspect of the present invention, the soybean is transgenic. In yet a further aspect, the transgenic soybean comprises an exogenous gene conferring herbicide resistance. In yet a further aspect, the transgenic soybean is resistant to glyphosate herbicides.

The present invention further provides a soybean meal, generated from a soybean capable of commercial yields, comprising at least about 56% protein on a dry weight basis, wherein the soybean has a yield, under standard agronomic practices, of at least about 30 bushels per acre.

The present invention further provides a soybean meal, generated from a soybean comprising a mean whole seed total protein plus oil content of at least about 64% on a dry weight basis, wherein the soybean is capable of commercial yields. The present invention further provides a soybean meal, generated from a soybean comprising a mean whole seed total protein plus oil content of at least than about 64% on a dry weight basis, wherein the soybean has a yield, under standard agronomic conditions, of at least about 30 bushels per acre.

The present invention further provides a soybean meal resulting from the processing of a high protein soybean variety, said soybean variety having a mean whole seed total protein content of greater than about 45% on a dry weight basis, and wherein the soybean variety is capable of commercial yields.

The present invention further provides a protein isolate and a protein concentrate prepared from the soybean meal of the present invention.

### DETAILED DESCRIPTION OF THE PREFERRED EMBODIMENTS

The present invention includes the use of a new soybean meal in animal and aquaculture feeding operations.

The following definitions are used herein:

Agronomically Elite: A soybean genotype that has many distinguishable traits, such as emergence, vigor, vegetative vigor, disease resistance, seed set, standability, and threshability, which allows a producer to harvest a product of commercial significance.

Commercial Yield: A yield of grain having commercial significance to the grower represented by an actual grain yield of at least 30 bushels per acre (Bu/A) as a mean measured over at least 14 environments, grown under standard agronomic practices.

Comparative Yield: A yield of grain, stated as a percentage of a yield of another soybean variety grown under comparative yield trial conditions. Conditions for comparative yield trials are well known in the art of soybean breeding. For example, a soybean variety having a yield of 43 Bu/A would have a comparative yield of 80% of an agronomically elite soybean variety having a yield of 54 Bu/A.

Dehulled Soybean Meal: A soybean meal having most of the hull fraction removed during the dehulling process step. The dehulled, solvent extracted soybean meal must not contain more than 3.5% fiber and typically contains between 48-50% protein at a 12% moisture basis.

Exogenous Protein: Protein that is not an intrinsic part of the soybean from which the soybean meal has been produced. Exogenous protein may be added to the meal or to the feed, in order to increase the protein concentration in the respective products.

Full Fat Soybean Meal: A soybean meal produced without extraction of oil.

High Fiber Soybean Meal: A soybean meal wherein the dehulling process has been omitted or minimized. Typical fiber levels in high fiber soybeans are 4-8% at 12% moisture basis.

Isonutritive diets: Animal diets formulated to have equal levels of nutrients, including energy, protein, and essential amino acids.

Isometric diet: Animal diets that are formulated to have the same levels of a particular ingredient. For instance, a hypothetical diet A containing 25% commodity soybean meal, and a hypothetical diet B containing 25% high protein soybean meal, are said to be isometric with respect to soybean meal.

Isonitrogenous diet: Animal diets that are formulated at the same levels of protein and essential amino acids.

Lodging Score: Lodging is rated on a scale of 1 to 9. A score of 1 indicates erect plants. A score of 5 indicates plants are leaning at a 45 degree(s) angle in relation to the ground and a score of 9 indicates plants are laying on the ground.

Oil content: Weight percentage of oil contained in soybean seed or soybean meal, stated on a dry basis.

Phenotype: The detectable characteristics of a cell or organism, which characteristics are the manifestation of gene expression.

Protein Content: Weight percentage of protein contained in soybean seed or soybean meal, stated on a dry weight basis unless noted otherwise.

Relative Maturity: The maturity grouping designated by the soybean industry over a given growing area This figure is generally divided into tenths of a relative maturity group. Within narrow comparisons, the difference of a tenth of a relative maturity group equates very roughly to a day difference in maturity at harvest.

Soybean Meal: A feed ingredient that is a product of processing soybean grain, wherein most of the oil (fat) is removed. The phrase “soybean meal,” as used in the context of this present invention, refers to a defatted, desolventized, toasted, and ground soybean material, to which no exogenous source of protein has been added.

Soybean Protein Isolate: The major proteinaceous fraction of soybeans, prepared from dehulled soybeans by removing the majority of non-protein components and containing not less than about 90% protein on a dry weight basis.

Soybean Protein Concentrate: A preparation from high quality soybean seeds, prepared by removing most of the oil and water soluble non-protein constituents and containing not less than about 65% protein on a moisture-free basis.

Standard Agronomic Practices: Those practices employed by a commercial grower, which would ensure at least an average yield for the defined region. Included in standard agronomic practices are planting, fertilization, weed control, insect control, disease control, and grain harvest.

### High Protein Soybean Varieties

The present invention provides high protein soybean meals derived from soybean varieties that are capable of commercial yields and have a protein content of at least about 45% on a dry weight basis. Additionally, the present invention provides soybean meal derived from soybean varieties that are capable of commercial yields, and have a high protein content without a corresponding reduction in seed oil. In particular, the present invention provides soybean meals having a protein content greater than at least about 58% protein on a dry weight basis, derived from soybean varieties with a mean whole seed total protein content of greater than about 45%. Such soybean varieties are characterized as being capable of a commercial yield. As used herein, a commercial yield is defined as a mean yield of at least about 30 bushels per acre, measured over at least 14 environments, and grown with standard agronomic practices.

The high protein soybean varieties of the present invention preferably further comprise a mean whole seed total protein plus oil content of greater than about 64%, about 66%, about 68%, or about 70% on a dry weight basis. In further embodiments of the present invention, the high protein soybean varieties have a mean whole seed total protein content on a dry weight basis of at least about 45% up to about 50%.

Examples of soybean varieties that are used in the context of the present invention are those having a mean whole seed total protein content of greater than about 45%, or a mean whole seed total protein plus oil content of about 64%. Most preferably such soybean varieties are capable of a commercial yield, such as, without limitation, soybean varieties 0008079, 0137335, 0137472, 0137441, and 0137810, as described by Byrum et al. (U.S. Published Application No. 20040060082).

Further examples of high protein soybean varieties used in the context of the present invention that have a capability for commercial yields are the soybean varieties DBL3404D0R, DCP2904B0R, DFN3204E0R, DFN2204D0R, DRM2004A0R, DOX2804E0R.

Additional examples of high protein soybean varieties preferably used in the context of the present invention, that are capable of commercial yields, are the soybean varieties EXP125A (designated as “Soybean variety 007583” in U.S. patent application Ser. No. 10/194,922, filed Jul. 11, 2002; American Type Culture Collection (ATCC) deposit number PTA-5764), EXP2702REN (designated as “Soybean variety 0137443” in U.S. patent application Ser. No. 10/745,299, filed on Dec. 23, 2003; ATCC deposit number PTA-5762), EXP2902REN (designated as “Soybean variety 0137400” in U.S. patent application Ser. No. 10/745,300, filed on Dec. 23, 2003; ATCC deposit number PTA-5763), EXP2303REN, and EXP3103REN.

One preferred aspect of the present invention is directed to a soybean meal generated from soybean varieties having the characteristics set forth above, and, in particular, from the specific soybean varieties set forth herein as examples. A further aspect of the present invention is directed to soybean meal generated from soybeans generated from tissue cultures of regenerable cells of the above mentioned high protein soybean varieties, which cultures regenerate soybean plants capable of producing seed expressing all the physiological and morphological characteristics of the variety. Such regenerable cells may include embryos, meristematic cells, pollen, leaves, roots, root tips or flowers, or protoplasts or callus, derived therefrom.

The soybean varieties listed above are for illustrative purposes and are not intended to limit the scope of the present invention. Other soybean varieties having a mean whole seed total protein content of at least about 45%, or a mean whole seed total protein plus oil content of at least about 64%, and which are capable of commercial yields, may be used to generate the soybean meal of the present invention.

In a further preferred aspect, the soybean variety of the present invention has a comparative yield of at least about 67% of an agronomically elite variety. More preferably, the comparative yield of the soybean variety used in the context of the present invention is at least about 70%; yet more preferably, the comparative yield is at least about 75%; at least about 80%; at least about 90%; and most preferably, at least about 95%.

### Soybean Processing

Many methods are known for the processing of raw soybeans into oil and meal. Illustrative soybean meal preparation processes include those taught in U.S. Pat. Nos. 4,992,294; 5,225,230; 5,773,051; and 5,866,192. Typically, commercial soybean processes include the receipt of the soybeans from the field by any conventional transport means, such as, for example, truck, barge, or rail car. The soybeans, typically received in a dirty and often wet condition, may be cleaned by being placed in contact with a vibrating screen, by which the soybeans are separated from non-soybean material, such as, for example, rocks, sticks, leaves, stems, dirt, weed seeds, and unwanted fragments of soybeans. The cleaned soybeans, in combination with the loose hulls that are not removed by the vibrating screen, are transferred to an aspirator in which most of the remaining loose hulls are removed by air. The soybeans are transferred to storage, and the loose hulls are collected as a by-product for further processing.

At this point in the processing, the soybeans typically contain about 12% by weight (wt %) water, but the actual water content of the soybeans may vary based on a host of different factors. If the water content of the soybeans is in excess of about 12 wt %, then the soybeans may be subjected to drying to reduce the water content below about 12 wt % prior to placing in storage. The control of the water content is essential to prevent mold and microbial contamination during storage.

The processing procedures from this point forward depend upon the desired end products. For example, the soybeans may be first dehulled using such conventional equipment as cracking rolls or hammer mills in combination with a conventional aspiration system. Alternatively, the hulls may not be removed prior to further processing (see, for example, U.S. Pat. No. 5,225,230). In order to deactivate antinutritional factors, such as trypsin inhibitors, the soybeans may be subjected to heat for a set period of time prior to cracking, grinding, or crushing. The soybeans are then crushed or ground into a meal using conventional equipment, such as grooved rollers.

For cracking processes, clean, dry, whole soybeans are fed to coarsely corrugated roller mills or “crackers.” These crackers can have one or more sets of rolls. Soybean pieces, called “cracks,” are formed. The goal of the cracking step is to maximize the pieces that are ¼^{th} to ⅛^{th} the size of the starting soybean, and minimize the formation of fines, which are pieces less than 1 mm in diameter.

From the cracking mills, particles of whole soybeans (i.e., cracks) are conveyed to multistage aspiration dehulling systems, which typically employ 1 to 3 stages. Each stage consists of an aspirator and a size screening system. At each stage, the fiber-rich “hulls” are first removed by means of a counter-current air stream and a cyclone. The heavier, fiber-lean, “meats” fraction is conveyed to a screening system that removes at least one additional fraction by size, and yields one stream for further aspiration. Alternatively, screening can be employed prior to aspiration. The “hulls” stream is typically combined with other soy byproducts and used as an animal feed ingredient. The dehulled “meats” are then dehulled again to less than about 3% crude fiber by mass (4.28% on a defatted, dry basis) using a 2 stage commercial pre-extraction process. However, the single stage systems can be employed to yield meats streams.

The resulting meats are then heat conditioned in a rotary or stack cooker. The residence times of the cracks are typically between about 20 and about 40 minutes. Discharge temperatures typically are in the range of 120 to 180° F. Lower conditioning temperatures may be employed if a greater fines production in the flaker is tolerable.

The conditioned meats are then fed to smooth roller mills called flakers. A force of greater than about 500 kPa-gauge (72.5 psig) are typically applied to the rolls. Flake thicknesses of less than about 0.75 mm (0.030″) are preferably produced in order to obtain maximum oil recovery in the subsequent oil extraction step. Optionally, the cracking and dehulling steps could be eliminated, or done subsequent to the conditioning step. An additional option would be to expand a percentage of the flaked soybeans to form “collets” prior to oil extraction. Other process variations include conditioning prior to the cracking step, and eliminating the dehulling step prior to oil extraction. A soybean meal of the present invention produced in a process having the variation of eliminating or reducing the dehulling step would be considered a high protein and high fiber soybean meal. A high fiber soybean meal would have a fiber content of between 4 and 8%. This product would be a desirable feed ingredient in a swine production operation.

The next step in the process of generating soybean meal is the extraction of oil. This extraction step is typically done using a lipophilic solvent, but may also be done by mechanical extraction. In this process, the soybean meal is contacted with a suitable solvent, e.g., hexane, to remove the oil to a content of typically less than about 1% by weight. One example of a conventional solvent extraction procedure is described in U.S. Pat. No. 3,721,569.

However, if a “full fat” soybean meal is desired, then the oil bearing meal is not subjected to oil (also known as fat or lipid) extraction. In this embodiment of the present invention, the resulting product would be a high protein, “full fat” soybean meal.

At this stage, the solvent extracted, defatted soybean meal typically contains about 30% solvent by weight. Prior to being used as an animal feed, the meal is typically processed through a desolventizer-toaster (DT) operation to remove residual solvent and to heat the protein fraction to inactivate trypsin inhibitors and other naturally occurring toxicants. Typically, steam contacts the soybean meal and the heat of vaporization released from the condensing steam vaporizes the solvent, which is subsequently recovered and recycled.

Alternatively, the soybean meal is defatted mechanically using, for example, a screw press. This mechanically extracted or “expeller” soybean meal typically contains between about 4 and about 8 wt % residual oil. If the intended use of the meal is as a feed supplement for ruminants, then the meal may first be heated and dried in a specified manner, such as that taught in U.S. Pat. No. 5,225,230, before oil is extracted mechanically. The defatted soybean meal is then dried and typically ground or pelletized and then milled into a physical state suitable for use as a food supplement or as an animal feed.

Further processing of the soybean or the meal may be done to make the resulting feed more palatable, available and/or digestible in animals. These processes include addition of enzymes or nutrients, and heat treating the meal. Additionally, further processing may be done to the meal, such as pellet and cub, to make it more compact and dense in distribution.

Further processing of the soybean meal can produce soybean flour, soybean protein concentrates, and soybean protein isolates that have food, feed, and industrial uses. Soybean flours are produced simply by grinding and screening the defatted soybean meal. Soybean protein concentrates, having at least about 65 wt % protein, are made by removing soluble carbohydrate material from defatted soybean meal. Aqueous alcohol extraction (60-80% ethanol) or acid leaching at the isoelectric pH 4.5 of the protein are the most common methods of removing the soluble carbohydrate fraction. A myriad of applications have been developed for soybean protein concentrates and texturized concentrates in processed foods, meat, poultry, fish, cereal, and dairy systems, any of which can be employed with the high protein soybean meal of the present invention.

Soybean protein isolates are preferably produced through standard chemical isolation, drawing the protein out of the defatted soybean flake through solubilization (alkali extraction at pH 7-10) and separation followed by isoelectric precipitation. As a result, isolates are at least about 90 wt % protein on a dry weight basis. They are sometimes high in sodium and minerals (ash content), a property that can limit their application. Their major applications have been in dairy substitution, as in infant formulas and milk replacers.

Soybean flours are often used in the manufacturing of meat extenders and analogs, pet foods, baking ingredients, and other food products. Food products made from soybean flour and isolate include baby food, candy products, cereals, food drinks, noodles, yeast, beer, ale, and the like.

The soybean meal of the present invention can be further processed into any of the products described herein. The advantages of using the high protein soybean meals of the present invention are the higher protein and lower carbohydrate contents, thus reducing the extent of processing to achieve the desired end products.

Soybeans additionally have many industrial uses. One common industrial usage for soybeans is the preparation of binders that can be used to manufacture composites, such as wood composites. Soybean-based binders have been used to manufacture common wood products such as plywood for more than 70 years. Although the introduction of urea-formaldehyde and phenol-formaldehyde resins has decreased the usage of soy-based adhesives in wood products, environmental concerns and consumer preferences for adhesives made from a renewable feedstock have caused a resurgence of interest in developing new soy-based products for the wood composite industry.

Preparation of adhesives represents another common industrial usage for the protein fraction from soybeans. Examples of soybean adhesives include soybean hydrolyzate adhesives and soybean flour adhesives. Soybean hydrolyzate is a colorless, aqueous solution made by reacting soybean protein isolate in a 5% sodium hydroxide solution under heat (120° C.) and pressure (30 psig). The resulting degraded soybean protein solution is basic (pH 11) and flowable (approximately 500 cps) at room temperature. Various adhesive formulations can be made from soy flour, with the first step commonly requiring dissolving the flour in a sodium hydroxide solution. The strength and other properties of the resulting formulation will vary depending on the additives in the formulation. Soy flour adhesives may also potentially be combined with other commercially available resins.

### Feed Formulations

The high protein soybean meal of the present invention is used in various feed formulations. In a preferred embodiment, the high protein soybean meal of the present invention is used in feed formulations for simple stomach animals, such as swine and poultry. Due to the higher protein content of the soybean meals of the present invention, inclusion rates are commonly reduced as compared to commodity soybean meal. Use of the high protein soybean meal of the present invention in feed formulations will reduce total soy protein, soy fiber, soy oligosaccharides, and potassium ion (K+) in the feed. Reducing these components may have benefit for young mammals and poultry that can not efficiently utilize soy fiber or soy protein sources. Additionally, the greater energy content in the high protein meal of the present invention as compared to commodity soybean meal will reduce the need for inclusion of exogenous fat and oil sources in poultry feed. This provides a potential benefit for poultry producers, enabling them to avoid the use of inconsistent feed grade sources of fat or oil. The combination of being able to reduce the total mass of soybean meal and fat or oil supplements, when using the high protein soybean meal of the present invention, will create more space in the feed formulation for additional ingredients. This characteristic of the soybean meal of the present invention provides the benefit to the animal producer and formulator of having more choices for the feed formulation.

Another characteristic of the high protein soybean meal of the present invention is the more consistent protein and energy quality as compared to commodity soybean meal. The more consistent protein and energy quality may reduce the use of other by-products, such as meat and bone meal and poultry by-product. This would reduce the need for storage bins for ingredients and hence reduce the cost of maintenance of such ingredient bins. Examples of the flexibility in feed formulation options when using high protein soybean meal of the present invention is demonstrated in the table below. The table shows the compositions of a typical corn-soybean meal formulation (Agri Stats 2001 Annual Analysis, Agri Stats Inc., Fort Wayne, Ind.), and three alternative formulations using high protein soybean meal of the present invention. The table illustrates the ability for a formulator to substitute bakery by-products or eliminate the use of meat and bone meal when using the high protein soybean meal of the present invention as an ingredient.

TABLE 1
Compositions of a typical corn-soybean meal formulation (Agri Stats
2001 Annual Analysis, Agri Stats Inc., Fort Wayne, Indiana), and
three alternative formulations using high protein soybean meal
of the present invention.

Broiler Grower
Formulations

| Ingredients | % | % | % | % |
|---|---|---|---|---|
| Corn | 60 | 65 | 65 | 60 |
| Soybean meal | 25 | — | — | |
| High protein soybean | — | 22 | 27 | 22 |
| meal | | | | |
| Meat and bone meal | 5 | 5 | — | — |
| Tallow | 4 | 2 | 2 | 4 |
| Bakery by-products | — | — | — | 8 |
| Micro ingredients | 6 | 6 | 6 | 6 |

The present invention is further described in the following Examples, which are offered by way of illustration and are not intended to limit the present invention in any manner. Standard techniques well known in the art, or the technique specifically described below, are utilized.

### EXAMPLE 1

This example describes the production of high protein soybeans useful in generating the high protein soybean meal of the present invention.

The six soybean varieties, exemplified below, were developed for high protein plus oil contents with equivalent yields to commercial varieties. The breeding and selection procedures followed those described by Byrum et al. U.S. Published Application No. 20040060082). Three separate yield trials were conducted under standard agronomic practices typically used by commercial seed producers, at different locations throughout Indiana, Illinois, and Iowa. In each location, yield measurements and lodging evaluations were made in addition to analyses for protein and oil. Comparisons were made to selected commercial varieties in each trial. The results of the trials are shown below in Tables 2-4.

TABLE 2
Yield trial evaluation of a high protein soybean variety DRM2004A0R, and selected
commercial soybean varieties. The results represent the means of 19 different
locations across the Midwestern United States.

| | RELATIVE | YIELD | LODGING | PROTEIN | OIL | PROT + OIL |
|---|---|---|---|---|---|---|
| VARIETY | MATURITY | (bu/A) | SCORE | (% dmb) | (% dmb) | (% dmb) |
| DRM2004A0R | 2.0 | 48.0 | 1.6 | 45.3 | 20.6 | 65.9 |
| ASGROW BRAND | 1.9 | 48.5 | 2.2 | 40.1 | 22.8 | 62.9 |
| AG1901 | | | | | | |
| PIONEER BRAND 91M90 | 1.9 | 47.3 | 1.8 | 41.2 | 20.9 | 62.1 |
| DEKALB BRAND DKB19- | 1.9 | 47.4 | 1.3 | 39.7 | 21.7 | 61.4 |
| 52 | | | | | | |
| SYNGENTA BRAND S19- | 1.9 | 48.5 | 1.2 | 40.3 | 21.3 | 61.6 |
| V2 | | | | | | |
| ASGROW BRAND | 1.9 | 49.5 | 1.5 | 39.7 | 20.9 | 60.6 |
| AG1903 | | | | | | |
| ASGROW BRAND | 2.0 | 48.0 | 1.8 | 41.2 | 22.0 | 63.2 |
| AG2001 | | | | | | |
| DEKALB BRAND DKB20- | 2.0 | 52.2 | 1.3 | 40.4 | 22.1 | 62.5 |
| 52 | | | | | | |
| PIONEER BRAND 92M00 | 2.0 | 46.9 | 1.6 | 40.5 | 21.8 | 62.3 |

TABLE 3
Yield trial evaluation of the high protein soybean varieties DOX2804E0R and DCP2904B04,
and selected commercial soybean varieties. The results represent the means of
25 different locations across the Midwestern United States.

| | RELATIVE | YIELD | LODGING | PROTEIN | OIL | PROT + OIL |
|---|---|---|---|---|---|---|
| VARIETY | MATURITY | (bu/A) | SCORE | (% dmb) | (% dmb) | (% dmb) |
| DOX2804E0R | 2.8 | 49.5 | 3.4 | 44.0 | 20.2 | 64.2 |
| DCP2904B0R | 2.9 | 50.0 | 2.7 | 45.5 | 19.5 | 65.0 |
| DEKALB BRAND DKB26- | 2.6 | 48.9 | 3.7 | 40.2 | 21.9 | 62.0 |
| 52 | | | | | | |
| PIONEER BRAND | 2.7 | 52.2 | 3.0 | 38.6 | 22.8 | 61.4 |
| 92M70 | | | | | | |
| ASGROW BRAND | 2.7 | 51.2 | 2.9 | 38.1 | 22.7 | 60.8 |
| AG2703 | | | | | | |
| ASGROW BRAND | 2.7 | 50.7 | 3.0 | 40.9 | 21.3 | 62.2 |
| AG2705 | | | | | | |
| PIONEER BRAND | 2.8 | 54.0 | 2.3 | 39.8 | 22.2 | 62.0 |
| 92M80 | | | | | | |
| DEKALB BRAND DKB28- | 2.8 | 53.0 | 3.1 | 39.5 | 21.9 | 61.4 |
| 53 | | | | | | |
| ASGROW BRAND | 2.8 | 52.6 | 2.9 | 39.9 | 21.7 | 61.5 |
| AG2801 | | | | | | |
| SYNGENTA BRAND | 2.8 | 51.3 | 4.6 | 42.2 | 20.0 | 62.2 |
| S28-L9 | | | | | | |

TABLE 4
Yield trial evaluation of the high protein soybean varieties DBL3204F0R,
DFN3204E04, and DBL3404D0R, and selected commercial soybean varieties. The results
represent the means of 31 different locations across the Midwestern United States.

| | | | | | OIL | PROT + OIL |
|---|---|---|---|---|---|---|
| | RELATIVE | YIELD | LODGING | PROTEIN | (% | (% |
| VARIETY | MATURITY | (bu/A) | SCORE | (% dmb) | dmb) | dmb) |
| DBL3204F0R | 3.2 | 47.2 | 1.9 | 44.3 | 20.5 | 64.8 |
| DFN3204E0R | 3.2 | 47.3 | 2.7 | 44.4 | 19.5 | 63.9 |
| DBL3404D0R | 3.4 | 48.0 | 3.3 | 45.3 | 20.1 | 65.4 |
| DEKALB BRAND DKB31-51 | 3.1 | 51.4 | 2.1 | 38.8 | 23.4 | 62.2 |
| ASGROW BRAND AG3202 | 3.2 | 52.2 | 3.0 | 39.6 | 21.8 | 61.4 |
| SYNGENTA BRAND S32-G5 | 3.2 | 48.5 | 2.1 | 36.1 | 22.7 | 58.8 |
| ASGROW BRAND AG3302 | 3.3 | 51.3 | 3.2 | 39.0 | 22.2 | 61.2 |
| ASGROW BRAND AG3305 | 3.3 | 53.1 | 2.3 | 35.3 | 22.8 | 58.1 |
| SYNGENTA BRAND S34-U4 | 3.4 | 51.4 | 3.4 | 38.6 | 21.7 | 60.3 |
| PIONEER BRAND 93M41 | 3.4 | 51.0 | 2.7 | 37.8 | 22.9 | 60.7 |
| ASGROW BRAND AG3401 | 3.4 | 53.6 | 3.4 | 40.5 | 21.6 | 62.1 |
| PIONEER BRAND 93M60 | 3.6 | 51.9 | 3.4 | 39.2 | 22.2 | 61.4 |

The results above exemplify high protein soybean varieties, having an oil plus protein content of at least about 64% and capable of commercial yields, which could be used in generating the high protein soybean meal of the present invention.

### EXAMPLE 2

This example describes the production of EXP125A soybeans used in preparing a high protein soybean meal of the present invention.

The EXP125A soybeans are described as “Soybean Variety 007583” in U.S. patent application Ser. No. 10/194,922, ATCC deposit number PTA-5764.

Yield trials were conducted to evaluate EXP125A, and other examples of high protein soybean varieties, EXP2702REN and EXP2902REN. The trials were conducted under standard agronomic practices typically used by commercial seed producers, across 14 different locations throughout Indiana, Illinois, and Iowa and in each location comparisons were made to selected commercial varieties. The results of the trials are shown below, in Table 5, with the yields expressed as averages across 14 locations. The results indicate that the high protein soybeans evaluated in these trials were capable of a commercial yield.

TABLE 5
Evaluation of high protein varieties EXP125A, EXP2702REN,
and EXP2902REN, and selected commercial varieties.
The results represent the means of 14 different trial
locations across the Midwestern United States.

| Type | Variety | Yield (bu/A) |
|---|---|---|
| High Protein | EXP125A | 47 |
| High Protein | EXP2702REN | 47 |
| High Protein | EXP2902REN | 46 |
| Commercial | Asgrow A2247 | 46 |
| Commercial | Asgrow A2553 | 53 |
| Commercial | Pioneer 92B23 | 48 |
| Commercial | Pioneer 92B35 | 48 |
| Commercial | NK24-L2 | 48 |

To generate the quantity of soybeans needed for processing into high protein meal and the subsequent feeding trials described herein, the EXP125A soybeans were grown under standard agronomic practices in different locations in the midwestern United States (Iowa, Illinois, and Indiana). The production encompassed a total of approximately 12,000 acres of commercial farmland. A total of approximately 14,500 tons of soybean grain was harvested from all locations. All grain produced was transported to a common commercial scale processing facility.

### EXAMPLE 3

This example describes the production of a high protein soybean meal at a commercial scale processing facility. All unit operations described below were performed using commercially available equipment.

High protein soybeans, as described in Example 2, were delivered via truck, to the commercial processing facility. The delivered moisture contents of the soybeans were in the range of 11-12%. The oil content was measured at 19.5 wt %, and the protein content was measured at 45.2 wt % (dry matter basis).

The soybeans were cleaned and then dried to an average starting moisture of 10.4 wt %. The cleaned and dried soybeans were then cracked using double cracking rolls.

The soybean cracks were then conveyed to the 2-stage aspiration system. The resulting hulls, recovered from the aspiration stream, had an average fat content of 0.84 wt %. The resulting meats were then dehulled to ultimately yield a defatted finished meal with 2.9 wt % crude fiber. The settings on the aspiration vacuum system were adjusted as necessary to optimize the hull separation from the meats.

The meats were then heat conditioned in a rotary conditioning system. The discharge temperature was maintained between 157.4 to 160.1° F., and the nominal residence time for the cracks was 30 minutes.

A drag conveyor moved the hot cracks from the discharge of the conditioner to the feeder of several flakers. A variety of makes and models of flakers were employed during the processing of the cracks. The resulting flakes from all flakers were less than 0.4 mm (0.016″) thick. Approximately 60% of the flakes produced were subsequently expanded, using an expander, to produce collets.

The flakes and collets were then solvent extracted with iso-hexane percolated through a 26 foot diameter fixed bottom extractor at a ratio of 0.7-0.8 lb solvent/lb whole beans. The mixed collet and flake bed depth was 8 feet. The solids residence time was typically 20 minutes. The extractor temperature was maintained between 132.4 and 140.0° F. The solids to solvent feed ratio, solids residence time, solvent drainage time, bed depth, and other extractor parameter settings were adjusted to optimize oil extraction, and were within the ranges typically employed by those skilled in the art.

The solvent extracted flakes and collets were desolventized using a 168 inch desolventizer-toaster (DT). The extracted soybean oil was desolventized by a sequence of two rising film evaporators followed by one oil stripper, in series. Operating conditions were those typical for a commercial soybean extraction facility, and well known to those of skill in the art.

The resulting soybean meal was dried to a moisture content of less than 12.5 wt %, and then cooled to less than 104° F. The soybean meal was then hammer-milled such that greater than 80% of a representative sample could pass through a U.S. #10 mesh screen.

Approximately 1140 metric tons of high protein soybean meal was produced as described above. Composite samples from each railcar loaded out were analyzed, and the results are shown below in Table 6. This meal was then used in feeding trials as described in the following Examples.

TABLE 6
Analysis of composite examples of high protein soybean meal
generated as described in Example 3.

| | | | Residual | Crude | |
|---|---|---|---|---|---|
| | Urease pH | Crude Protein | Crude | Fiber | Moisture |
| | rise¹ | (%) | Fat (%) | (%) | (%) |
| Minimum | 0.02 | 52.8 | 0.9 | 2.6 | 11.4 |
| Average | 0.05 | 53.6 | 1.0 | 2.9 | 12.0 |
| Maximum | 0.11 | 54.4 | 1.2 | 3.4 | 12.6 |

¹Urease pH rise is an indicator of the extent of protein denaturation taken place during the toasting operation. The pH rise is directly proportional to the amount of nondenatured urease.

### EXAMPLE 4

This example describes the determination of true metabolizable energy (TME) of the high protein soybean meal produced on a commercial scale, as described in Example 2.

A metabolic study was designed to determine the true metabolizable energy of the high protein soybean meal. Thirty six single comb white leghorn roosters (Hy-Line breed), at 44 weeks of age, were allotted to 3 treatments in a 3×3 Latin square design. The 3 treatments were:
Control—yellow corn for endogenous energy determination
Soybean meal A—pilot scale processed high protein soybeans
Soybean meal B—pilot scale processed commercial soybeans

Prior to the study, the roosters were randomly placed in individual metabolic cages and fasted for 30 hours. After the second day, each rooster was fed 35 grams of the corresponding feed treatment or control, and the excreta was collected in a stainless steel pan for 48 hours. The procedure was repeated 3 times.

The collected excreta was individually weighed, dried, and weighed again to calculate the moisture content. Three samples were then pooled randomly for gross energy (GE) determination using a bomb calorimeter (Parr Instrument Co., Moline, Ill.). Pooled excreta for each treatment was then ground to a powder in a standard Wiley mill, and approximately 1 gram of each powder was pelleted using a Parr pellet press (Parr Instrument Co., Moline, Ill.). The pellet samples were then placed in an adiabatic oxygen bomb (Parr Instrument Co., Moline, Ill.) and the gross energy determined. Analyses of pooled samples were done in duplicate.

The gross energy of the different soybean meals were determined by a similar procedure as the excreta. Ground samples of the meal were pelleted, using the same equipment as described above. The pelleted samples were then placed in the same adiabatic oxygen bomb, and the gross energy determined as described above. Analyses of pooled samples were done in duplicate.

The true metabolizable energy (TME) was calculated using the following equation:

TME=(grams feed×(GE feed)×(grams collected excreta×GE collected excreta)—(endogenous GE))/grams feed

As used herein, endogenous GE is defined as the gross energy of collected fecal sample from a rooster fed a control feed (97% yellow corn and 3% vitamins/minerals).

Additionally, analyses for protein, oil, crude fiber, neutral detergent fiber (NDF), acid detergent fiber (ADF), ash, and amino acids profiles were done and compared to values from a standard soybean meal as referenced in the National Research Council (NRC) (Nutrient Requirement for Poultry (1994) and Nutrient Requirement for Swine (1998)). All analyses followed protocols set forth by the AOAC® Official Methods^{SM} (AOAC® International, Gaithersburg, Md.). Briefly, crude protein analysis followed AOAC® Official Method 990.03 (2000); crude fiber followed AOAC® Official Method 978.10 (2000); ash followed AOAC® Official Method 942.05 (2000); and amino acid profiles followed AOAC® Official Method 982.30 E (a, b, c), CHP. 45.3.05 (2000). Analyses for NDF and ADF followed AOAC® 56:1352-1356 (1973) and AOAC® Official Method 973.18 (A-D) (2000), respectively, with some modification.

The results shown below in Table 7 indicate that the TME of high protein soybean meal is 175 kcal/kg greater than the TME in regular soybean meal (2660 vs. 2485 kcal/kg). Additionally, there is a greater increase in concentrations of arginine and valine as compared to the increase in protein. Therefore, the quality of amino acids is another distinguishing feature of the high protein soybean meal.

TABLE 7
Comparative analysis of high protein soybean meal of the present
invention and commercial (regular) soybean meal.

| | |
|---|---|
| HP SBM | Regular SBM^{a} |

Standard Analysis, % (90% Dry Matter)

| | | |
|---|---|---|
| Protein | 55.9 | 48.5 |
| Oil | 1.2 | 1.0 |
| Crude Fiber | 2.8 | 3.9 |
| NDF | 6.9 | 8.9 |
| ADF | 2.8 | 5.4 |
| Ash | 6.4 | 5.7 |

Energy, kcal/kg

| | | |
|---|---|---|
| Poultry TME | 2,660 | 2,485 |

Amino Acid Content, % (88% Dry Matter)

| | HP SBM | Regular SBM |
|---|---|---|
| Lysine | 3.34 | 2.96 |
| Methionine | 0.75 | 0.67 |
| Cysteine | 0.81 | 0.72 |
| Threonine | 2.05 | 1.87 |
| Tryptophan | 0.78 | 0.74 |
| Arginine | 4.12 | 3.48 |
| Isoleucine | 2.50 | 2.12 |
| Leucine | 4.20 | 3.74 |
| Valine | 2.67 | 2.22 |
| Histidine | 1.48 | 1.28 |
| Phenylalanine | 2.80 | 2.34 |
| Tyrosine | 1.91 | 1.95 |
| Glycine | 2.26 | 2.05 |
| Serine | 2.36 | 2.48 |
| Alanine | 2.25 | — |
| Aspartate | 6.35 | — |
| Glutamate | 10.48 | — |

^{a}Regular soybean meal composition data is from NRC, Nutrient Requirement for Poultry (1994) and Swine (1998).

### EXAMPLE5

This example describes a feeding trial with broilers, evaluating the high protein soybean meal generated as described in Example 2.

A controlled floor pen study using a total of 960 male broilers (hereafter referred to as “birds”) was conducted to evaluate the nutritional value of the high protein soybean meal (HPSBM), as compared to commodity soybean meal (SBM). One half (480) of the birds used were Cobb 500 (Cobb-Vantress, Siloam Springs, Ark.) and the other half were Ross 308 (Aviagen, Huntsville, Ala.). The birds were randomly allotted to the 3 treatments described in the table below.

TABLE 8
Description of treatments in broiler feeding
trial described in Example 5.

| | Treatment 1 | Treatment 2 | Treatment 3 |
|---|---|---|---|
| | SBM-Control | HPSBM-I | HPSBM-II |
| Description | Commodity | Diet was formulated | Isometric diet as |
| | Soybean Meal | to same protein and | treatment 1 |
| | (SBM) | amino acid level as | replacing SBM |
| | | treatment 1. | usage with HPSBM |
| | | Assumes equal ME | equally |
| | | between HPSBM | |
| | | and SBM | |
| Major | Corn, SBM, | Corn, HPSBM, | Corn, HPSBM, |
| ingredients | tallow | tallow | tallow |

Nutrient Requirements (meet average nutrient requirement of Agri
Stats 2001 Annual Analysis, Agri Stats Inc., Fort Wayne, Indiana)

| | Starter | Grower | Finisher |
|---|---|---|---|
| ME, kcal/lb | 1,397 | 1,434.00 | 1,465 |
| Crude Protein, % | 22.7 | 20.3 | 16.8 |
| Lysine, % | 1.32 | 1.15 | 0.90 |

The birds were fed starter, grower, and finisher diets formulated to the treatment strategy listed above from day 1 through day 42. Each diet was fed for 14 days. The results shown in the table below indicate that the birds fed HPSBM-II had a significantly greater weight gain and better feed conversion rate as compared to the other 2 diets. The birds fed HPSBM-I diets grew slightly less than birds fed control feeds (P>0.05). The feed conversion rate, however, is 4.2 points better than the control diets (1.748 vs. 1.790 for HPSBM-I and SBM Control, respectively). These results indicate that the energy of HPSBM is higher than commercial SBM and therefore the birds fed the HPSBM-I diets grew at a similar rate but gain weight more efficiently. These results corroborate the results of the TME determination described in Example 3.

TABLE 9
Results of broiler feeding trial described in Example 5.

| Treatments | Average daily gain, g | Feed:Gain Ratio |
|---|---|---|
| SBM-Control | 55.92 | 1.790 |
| HPSBM-I | 55.06 | 1.748 |
| HPSBM-II | 57.66* | 1.698* |

*P < .05

### EXAMPLE 6

This example describes a commercial scale broiler feeding trial comparing the performance of a commodity soybean meal having 48% protein (48% SBM) with the high protein soybean meal (HPSBM) prepared as described in Example 2.

This study was conducted at multiple commercial farms in the southeastern United States. The commercial barns used in the study ranged in age from 1 to 25 years old, with all having heating and ventilation provided. Each commercial barn contained between 15,000 and 18,000 birds. Clean water and fresh feed were provided to birds ad libitum. Routine health and management programs were used in all commercial farms without additional modifications. Barns were randomly assigned for feeding the control rations containing commodity soybean meal or the rations containing high protein soybean meal.

Four different phases of feed corresponding to starter, grower, withdraw 1, and withdraw 2, were formulated according to standard industrial practices as outlined in Agri Stats Report 2003 (Agri Stats, Fort Wayne, Ind.). The base ingredients in the feeds were corn and soybean meal, with the balance of the formulation consisting of a few common by-products from bakeries and rendering plants. Feeds at each phase were formulated at equivalent levels of energy, protein, and essential amino acids, using either commodity soybean meal or high protein soybean meal. The pooled data is shown in the table below.

TABLE 10
Results from commercial feeding trial using the high protein soybean
meal of the present invention, as described in Example 6.

| | | | | | calories |
|---|---|---|---|---|---|
| | # of | Livability, | 1st week | Feed:Gain | required for |
| | birds | % | mortality, % | ratio | a 5 lb. bird |
| HPSBM | 819,500 | 96.75 | 0.87 | 1.86 | 2,587 |
| 48% SBM | 564,600 | 96.39 | 1.29 | 1.90 | 2,620 |

These results indicate that the birds fed with the HPSBM had a slightly better livability (+0.36) and an improved feed:gain ratio (4 points). These results indicate that under the isocaloric and isonitrogenous conditions of this study, the HPSBM demonstrated improved growth performance when compared to standard commercial soybean meal.

### EXAMPLE7

This example describes the protein and amino acid digestibility of high protein soybean meal generated in a pilot plant as compared to commodity soybean meal, generated in a pilot plant and at commercial scale.

One hundred and eighty male Ross 308 broilers were used in an experiment to determine the amino acid (AA) digestibility of pilot plant processed high protein soybean meal (HPSBM). The experiment was conducted as a randomized complete block design with 5 dietary treatments and 6 replicates per treatment. Each treatment replicate consisted of 2 pens with 3 birds per pen. Common corn-soybean meal, starter, and grower diets (formulated at industry average level) were fed for 26 days. At 26 days of age, the birds were weighed and sorted to equalize the average weight among replicates. Treatment diets were started at 26 days of age and fed for 4 days. Birds had ad libitum access to feed and water. At 29 days of age, fresh excreta were collected for determination of energy digestibility and amino acid digestion.

All test diets, as shown in Table 1, contained the same concentration of all ingredients, with the exception of soybean meal source. Chromic oxide and titanium were added to all diets as indigestible markers.

Treatment assignments for the soybean meals of this study are described below:
1. Commodity soybeans processed at a commercial crush plant.
2. Commodity soybeans processed at pilot plant scale (same soybean source as Treatment 1).
3. High protein soybeans processed at pilot plant scale (meal contains 4,900 trypsin inhibitor units, and 0.11 urease pH rise).
4. High protein soybeans processed at pilot plant scale (meal contains 6,800 trypsin inhibitor units, and 0.47 urease pH rise).

Treatments 3 and 4 represent samples taken at different times during the same processing run. Excreta samples from the 2 pens that made up a replicate of a treatment were combined; frozen, lyophilised, ground, and analyzed for chromic oxide and amino acids. Pen temperatures were controlled at 65+/−2° F. and a schedule of 23 hour lighting was used for the entire experiment, with the 1 hour dark period starting at midnight. Each pen consisted of 3 birds with a growing density of 0.67 square foot per bird.

The data were summarized by comparing replicate treatment means and statistical analysis of variance for each of the measurements was performed using General Linear Models (GLM) procedure of SAS (SAS Institute Inc., Cary, N.C.).

TABLE 11
Ingredient composition of test diets

| | Commercial | Pilot | Pilot | |
|---|---|---|---|---|
| | Normal | Normal | High protein | |
| | SBM | SBM | SBM | |
| Ingredients | (1) | (2) | (3) | (4) |
| Commodity SBM¹ | 99.203 | — | — | — |
| Pilot Commodity SBM² | — | 99.203 | — | — |
| Pilot HPSBM³ | — | — | 99.203 | — |
| Pilot HPSBM⁴ | — | — | — | 99.203 |
| Salt | 0.372 | 0.372 | 0.372 | 0.372 |
| Poultry trace mineral | 0.050 | 0.050 | 0.050 | 0.050 |
| Poultry vitamin | 0.125 | 0.125 | 0.125 | 0.125 |
| Titanium dioxide | 0.100 | 0.100 | 0.100 | 0.100 |
| Chrornic oxide | 0.150 | 0.150 | 0.150 | 0.150 |

¹Commodity soybean meal (SBM), processed at a commercial crush plant.
²Pilot plant scale produced commodity SBM (same soybean source as Treatment 1).
³Pilot plant produced HPSBM having 4,900 trypsin inhibitor units, and 0.11 urease.
⁴Pilot plant produced HPSBM having 6,800 trypsin inhibitor units, and 0.47 urease.

The digestibility data is shown in Table 12. The digestibility of cysteine was higher (P<0.04) for the two HPSBM treatments as compared to the two commodity SBMs (treatments 3 and 4 vs. treatments 1 and 2, respectively). However, for all other amino acids the HPSBM and commodity SBM had equivalent (P>0.05) digestibility. There was no difference (P>0.06) in digestibility of the commodity SBM processed at commercial scale and that of the commodity SBM processed at pilot plant scale (treatment 1 and treatment 2, respectively). Additionally, there was no difference (P>0.15) for the mean of all the SBM processed at pilot plant scale, for any amino acid (treatments 2, 3, and 4). The mean of the two HPSBM (treatments 3 and 4) was higher (P<0.04) in digestibility for methionine, cysteine, valine, and isoleucine than the pilot scale processed normal meal (treatment 2). All other amino acids had equal (P>0.04) digestibilities. There was no difference (P>0.15) in digestibility between the two SBMs processed at pilot plant scale.

TABLE 12
The amino acid digestibility¹ of commodity SBM and HPSBM

| | Soybean Meal Type | | | |
|---|---|---|---|---|
| | Commodity | Commodity- | | HP- |
| | Commercial² | pilot³ | HP-Pilot-4.9⁴ | Pilot-6.8⁵ |
| Amino Acids | (1) | (2) | (3) | (4) |
| Methionine | 89.06 | 87.26 | 91.35 | 89.04 |
| Lysine | 89.40 | 87.99 | 90.67 | 89.40 |
| Cystine | 79.09 | 74.61 | 82.23 | 78.70 |
| Valine | 85.39 | 83.37 | 87.81 | 85.60 |
| Threonine | 81.42 | 79.48 | 84.08 | 81.11 |
| Histidine | 88.69 | 88.08 | 90.49 | 88.81 |
| Phenylalanine | 87.32 | 86.12 | 89.54 | 87.36 |
| Isoleucine | 86.99 | 85.06 | 89.38 | 87.49 |
| Leucine | 87.00 | 85.67 | 89.45 | 87.55 |
| Arginine | 89.09 | 87.70 | 88.19 | 86.15 |
| Tryptophan | 90.86 | 90.91 | 92.53 | 91.07 |

¹From excreta collected from the lower ileum of 30 day old Ross 308 broilers.
²Commodity soybeans processed at the commercial crush plant.
³Pilot plant scale crushed commodity soybean (same soybean source as Treatment 1).
⁴Pilot plant scale crushed HPSBM (4,900 trypsin inhibitor units, and 0.11 urease rise).
⁵Pilot plant scale crushed HPSBM (6,800 trypsin inhibitor units, and 0.47 urease rise).

1. A soybean meal comprising at least 58% protein on a dry weight basis, wherein no exogenous source of protein has been added, and wherein said meal is generated from a soybean capable of commercial yields.

2. The soybean meal of claim 1 wherein the soybean has a protein content of at least 45% on a dry weight basis.

3. The soybean meal of claim 1 wherein the soybean has an oil plus protein content of at least 64% on a dry weight basis.

4. The soybean meal of claim 1, wherein the meal comprises at least 60% protein on a dry weight basis.

5. The soybean meal of claim 1, wherein the meal comprises at least 62% protein on a dry weight basis.

6. A soybean meal resulting from the processing of a high protein soybean variety, said soybean variety having a mean whole seed total protein content of greater than about 45% on a dry weight basis, and wherein the soybean variety is capable of commercial yields.

7. The soybean meal of claim 1, wherein the soybean is transgenic.

8. The soybean meal of claim 7, wherein the transgenic soybean comprises an exogenous gene conferring herbicide resistance.

9. The soybean meal of claim 8, wherein the transgenic soybean is resistant to glyphosate herbicide.

10. A feed containing the soybean meal of claim 1.

11. A protein isolate prepared from the soybean meal of claim 1.

12. A protein concentrate prepared from the soybean meal of claim 1.

13. A method of feeding an animal comprising incorporating into a feed ration a soybean meal comprising at least 58% protein on a dry weight basis, wherein no exogenous source of protein has been added, and wherein said meal is generated from a soybean capable of commercial yields.

14. The method of claim 13, wherein the meal comprises at least 60% protein on a dry weight basis.

15. The method of claim 13, wherein the meal comprises at least 62% protein on a dry weight basis.

16. The method of claim 13, wherein the animal is selected from the group consisting of poultry, swine, bovine, and companion animal.

17. The method of claim 13, wherein the animal is poultry.

18. A soybean meal comprising at least 58% protein on a dry weight basis, wherein no exogenous source of protein has been added, and wherein said meal is generated from a soybean variety comprising ATCC deposit number PTA-5764.

19. A method of processing a soybean comprising providing a soybean comprising a protein content of at least 45% on a dry weight basis, and wherein the soybean is capable of commercial yields, processing the soybean to produce an oil fraction and a meal fraction.
